SEQUENCE LISTING (8 sequences)

SEQ ID NO: 1 - DNA, 21 bp, synthetic construct
  source 1..21: mol_type = other DNA
gaagccaggtgtctccaagag

SEQ ID NO: 2 - DNA, 21 bp, synthetic construct
  source 1..21: mol_type = other DNA
gtggatgtcctttaccgtcgt

SEQ ID NO: 3 - DNA, 20 bp, synthetic construct
  source 1..20: mol_type = other DNA
gccgggaatgatgagaacta

SEQ ID NO: 4 - DNA, 20 bp, synthetic construct
  source 1..20: mol_type = other DNA
ggaccgtccactgtcacttt

SEQ ID NO: 5 - DNA, 24 bp, synthetic construct
  source 1..24: mol_type = other DNA
ctgcccagaagaatatgtatcacc

SEQ ID NO: 6 - DNA, 23 bp, synthetic construct
  source 1..23: mol_type = other DNA
gaagcaaagtttcctccaagacc

SEQ ID NO: 7 - DNA, 20 bp, synthetic construct
  source 1..20: mol_type = other DNA
tgaggaccctctctctgctc

SEQ ID NO: 8 - DNA, 20 bp, synthetic construct
  source 1..20: mol_type = other DNA
aggtagcgccggagtctacc

(19) United States
(12) Patent Application Publication
(10) Pub. No.: US 2025/0256010 A1
(43) Pub. Date: Aug. 14, 2025

(54) COMPOSITION FOR REGENERATING MASTOID BONE USING IN VIVO ONE-STEP STEM CELL DIFFERENTIATION

(71) Applicant: AJOU UNIVERSITY INDUSTRY-ACADEMIC COOPERATION FOUNDATION, Suwon-si, Gyeonggi-do (KR)

(72) Inventors: Yun-Hoon CHOUNG, Seoul (KR); Hantai KIM, Hwaseong-si, Gyeonggi-do (KR); Sunghee PARK, Hwaseong-si, Gyeonggi-do (KR); Oak-Sung CHOO, Seongnam-si, Gyeonggi-do (KR)

(73) Assignee: AJOU UNIVERSITY INDUSTRY-ACADEMIC COOPERATION FOUNDATION, Suwon-si, Gyeonggi-do (KR)

(21) Appl. No.: 18/686,228

(22) PCT Filed: Aug. 19, 2022
(86) PCT No.: PCT/KR2022/012390

§ 371 Date: Feb. 23, 2024

(30) Foreign Application Priority Data
Aug. 23, 2021 (KR) 10-2021-0110755

(51) Int. Cl. A61L 27/38; A61L 27/08; A61L 27/18; A61L 27/36; A61L 27/56; C08L 67/04

(52) U.S. Cl. CPC A61L 27/3804; A61L 27/08; A61L 27/18; A61L 27/3616; A61L 27/56; C08L 67/04; A61L 2430/02

(57) ABSTRACT

A composition for regenerating the mastoid bone based on in vivo one-step stem cell differentiation is disclosed. The composition contains a three-dimensional porous scaffold and adipose tissues as active ingredients. The three-dimensional porous scaffold, adipose-derived cells, and plasma have the effect of significantly increasing bone regeneration for mastoid bone defects. The adipose tissues and whole plasma obtained as by-products during mastoid surgery are used for bone regeneration, and, thus, an effect of regenerating mastoid bone can be conveniently achieved in a one-step manner upon the surgery.

### TECHNICAL FIELD

The present invention relates to mastoid bone regeneration based on in vivo one-step stem cell differentiation technology, and more particularly to a composition for mastoid bone regeneration based on in vivo one-step stem cell differentiation technology, comprising a three-dimensional porous scaffold and adipose tissues as active ingredients.

### RELATED ART

Surgical treatments for chronic otitis media or otitis media cholesteatomatica include mastoidotomy and tympanoplasty. Tympanoplasty involves replacing the damaged ossicles and reconstructing the damaged eardrum, which is associated with improvement of hearing. Mastoidectomy involves the removal of all of the air cells of the mastoid that connects to the middle ear cavity and aims to prevent recurrence by removing all of the inflammatory tissues, or cholesteatomas, that fill the mastoid cavity.

When a mastoidectomy is performed, in which all of the lesions and the air cells of the mastoid cavity are removed with a drill, a space is left in the mastoid cavity that was originally filled with normal air cells. There is no consensus on whether this space should be left alone or filled with something else. Some argue that since the mucous membrane has been surgically removed, the mastoid cavity cannot perform its natural function of buffering and producing gas, and therefore, this empty space may put more strain on the middle ear, it is better to fill the space with other material and close it off, while others argue that even though the mucous membrane is damaged, it is better to leave it open because it can still contain gas and provide gas to the middle ear cavity. These two concepts are completely opposite and should be considered in relation to the function of the ear canal, but no clear conclusion has been reached.

This debate would not exist if the tissue in the mastoid cavity that was surgically removed could be regenerated or repaired in the form of original tissue of the mastoid cavity, which contained many air cells. If regeneration and repair were possible, it would be better to leave the mastoid cavity rather than fill it with external material. Unfortunately, however, it is virtually impossible for a surgically removed mastoid space to regain its original level of function.

Although it is concluded that, if the functional recovery of the surgical site in the mastoid cavity cannot be expected, the space may put a strain on the middle ear cavity, and closure of the space may be advisable, there may be another problem attributable thereto. Until now, mastoid cavity closure has been performed with autologous bone powder, cartilage, muscle, or synthetic bone. However, there are obvious limitations, for example, autologous bone powder or cartilage is not enough to fill the space, and synthetic bone is basically an external material, so problems such as infection may occur.

The ideal solution to this problem would be to regenerate the cut mastoid cavity with a porous structure similar to that of the original mastoid air cells. In other words, if the mastoid bone is closed with an appropriate material, but the material is similar to the original form of the mastoid bone, which is porous, restoration of the original physiological function of the mastoid can be expected, thus resolving both of the above arguments.

Tissue engineering is a technology that can enhance the regeneration of biological tissues based on main components: cells, scaffolds, and signaling factors. A three-dimensional scaffold is a space in which cells can attach and maintain their ability to differentiate and proliferate, and the inventors of the present invention believe that a three-dimensional scaffold can be used to restore the space removed by mastoidectomy to its original form. Polycaprolactone (PCL), a synthetic polymeric material, is an FDA-approved material that has been widely used in tissue engineering due to its low cost compared to natural polymeric scaffolds and the great advantage of being able to modify its molecular structure and molecular weight to control its physical and mechanical properties and biodegradability. Recently, it was reported that PCL/alginate and PCL/alginate/BMP-2 scaffolds coated with umbilical cord serum were effective for mastoid bone regeneration by implantation into the cavity after cutting off mastoid bone (Jang, Chul Ho et al, International journal of pediatric otorhinolaryngology 78:1061-1065, 2014; Jang, Chul Ho et al. in vivo 30:835-8390, 2016). However, it is difficult to expect mastoid bone regeneration by simply placing PCL into the surgical site.

Accordingly, the inventors of the present invention have made an effort to develop a method for restoring the removed mastoid cavity to its original form after mastoidotomy, which is performed for the surgical treatment of chronic otitis media or otitis media cholesteatomatica, and have confirmed that when the adipose tissue removed during mastoidotomy is re-implanted into a three-dimensional scaffold, the lost mastoid bone is effectively regenerated, and have completed the present invention.

### SUMMARY OF INVENTION

It is an object of the present invention to provide a composition for mastoid bone regeneration that can restore mastoid bone removed during mastoidectomy in one step.

To achieve the above objective, the present invention provides a composition for mastoid bone regeneration comprising a three-dimensional porous scaffold and adipose tissues as active ingredients.

### BRIEF DESCRIPTION OF DRAWING

FIG. 1 illustrates the results of stemness of cells cultured with mouse subcutaneous adipose tissue-derived SVF.

FIG. 2 shows the results of osteogenic promotion of adipose tissue-derived stem cells by plasma component treatment (CM, control medium; CMP, control medium with platelet poor plasma; CMR, control medium with platelet rich plasma; CMWP, control medium with whole plasma; OM, osteogenic medium; OMP, osteogenic medium with platelet poor plasma; OMR, osteogenic medium with platelet rich plasma; OMWP, osteogenic medium with whole plasma).

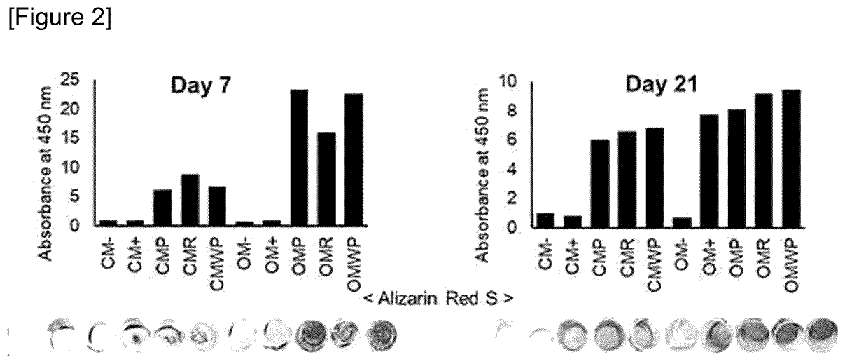

FIG. 3 shows the CT images of 2, 4, and 7 months after adipose transplantation to mouse mastoid bone defect models, and the results of analysis of H&E and immunostaining thereof (PCL, polycaprolactone; PRP, platelet rich plasma; WP, whole plasma; GO, graphene oxide; CTL, control; PF, polycaprolactone+adipose; PFP, polycaprolactone+adipose+platelet rich plasma; PFW, polycaprolactone+adipose+whole plasma; PGFW, polycaprolactone+graphene oxide+adipose+whole plasma).

FIG. 4 shows the results of tissue staining by group quantified by Image J by DAB (3,3′-diaminobenzidine) staining of osteocalcin (OCN) and bone sialo-protein (BSP), which are used as markers of osteogenic differentiation after adipose transplantation into a mouse mastoid bone defect model (OCN, osteocalcin; BSP, bone sialo-protein; PCL, polycaprolactone; PF, polycaprolactone+adipose; PFP, polycaprolactone+adipose+platelet rich plasma; PFW, polycaprolactone+adipose+whole plasma; PGFW, polycaprolactone+graphene oxide+adipose+whole plasma).

### DETAILED DESCRIPTION OF THE INVENTION AND PREFERRED EMBODIMENTS

Unless otherwise defined, all technical and scientific terms used herein have the same meaning as commonly understood by a person skilled in the art. In general, the nomenclature used herein is well known and in common use in the art.

The present invention has developed a method for restoring the removed mastoid cavity to its original form after mastoidotomy, which is performed for surgical treatment of chronic otitis media or otitis media cholesteatomatica, and it is difficult to expect the mastoid bone regeneration effect by injecting only a PCL scaffold, which is a known method, into the mastoid surgery site. The inventors of the present invention expected that the mastoid bone regeneration effect would be improved if stem cells existing in adipose tissue are used in the PCL scaffold. In particular, when mastoidectomy is performed, some adipose tissue around the ear is also removed, and it was found that if some of the removed adipose tissue is harvested and reimplanted into a three-dimensional scaffold, it can be regenerated into appropriate mastoid bone tissue.

Furthermore, implanting subcutaneous adipose tissue into a three-dimensional scaffold and treating appropriate growth factors may lead to more successful mastoid bone regeneration. In particular, plasma-derived growth factors are very effective. Platelet rich plasma contains many of these growth factors and is already used effectively in the treatment of musculoskeletal disorders. If successful mastoid bone regeneration can be achieved, it may ultimately overcome the disadvantages of open cavity mastoidectomy, which is a commonly performed mastoidectomy procedure. It is a surgical technique in which the posterior wall of the ear canal is removed together with the mastoid air cells during surgery, which is effective because it provides a good surgical view and reduces the chance of recurrence. However, when the posterior wall of the ear canal is removed, the spaces of the ear canal and the mastoid cavity are left as one open cavity after surgery. This results in a wide cavity inside the ear after surgery, which causes a number of problems: the patient needs to clean the ear regularly because the earwax does not naturally come out, and if the patient needs to use hearing aids due to hearing loss, it is very uncomfortable to wear hearing aids because of the large space in the ear. Therefore, due to these disadvantages, despite being a very good procedure in terms of recurrence prevention, it is necessary to be cautious in choosing the procedure. However, if the mastoid bone can be successfully regenerated using the mastoid bone regeneration composition of the present invention, this cavitation problem can be solved by regenerating the lost mastoid bone even after an open cavity mastoidotomy is performed. That is, this ultimately reduces the risk of recurrence while also resolving postoperative discomfort, which can be expected to provide optimal treatment results for patients.

Accordingly, the present invention relates to a composition for mastoid bone regeneration comprising a three-dimensional porous scaffold and adipose tissues as active ingredients.

In the present invention, the scaffold may be polycaprolactone (hereinafter, referred to as “PCL”) or graphene, and a scaffold comprising a mixture of polycaprolactone and graphene may also be used, preferably polycaprolactone.

The adipose tissue used in the present invention may be characterized as patient-derived autologous adipose tissue, and the patient-derived autologous adipose tissue may be, but is not limited to, adipose tissue around the ear that is removed during mastoidectomy.

In one aspect of the present invention, it has been confirmed that bone tissue is regenerated as when adipose tissue-derived stem cells are used, even when using adipose tissues rather than adipose tissue-derived stem cells. Therefore, according to the present invention, the mastoid bone regeneration effect can be achieved by transplanting the adipose tissue around the ear that is removed during mastoidectomy in one step without additional culture.

It is also economical because there is no need to grow stem cells in expensive culture media.

In the present invention, the adipose tissue transplanted into the mastoid bone is preferably cut into 40-80 mm³ (40-80 μL) pieces.

In one aspect of the present invention, a combination of PCL, a three-dimensional porous scaffold, adipose-derived cells, and plasma-derived growth factors is implanted in the space after a mastoid cavity defect caused by surgery for the purpose of mastoid bone regeneration, in terms of tissue engineering, and the regeneration effect of mastoid bone is confirmed. In particular, this present invention confirmed that plasma is as effective as platelet-rich plasma in regenerating mastoid bone. Platelet-rich plasma requires a process of collecting the patient's own blood and centrifuging it at high speed, but plasma does not require such a centrifugation process, so it has a great advantage that it can be performed in one step during the patient's surgical procedure without going through a separate process. In the present invention, it was confirmed that the process of mastoid bone closure can be carried out simultaneously without a separate procedure in the actual surgical process of the patient by using a porous scaffold, adipose tissue, and the patient's whole blood.

In the present invention, the composition may be characterized as further comprising plasma, in particular autologous plasma. The autologous plasma may be taken from whole blood that is removed during a mastoidectomy in a patient, and may be characterized in that it is whole plasma.

Since the bone in the mastoid is porous with holes therein, like a sponge, rather than a dense bone, it is very important to apply a dose that does not exceed the volume of the mastoid, as bone regeneration does not need to be dense.

The content of the composition of the present invention varies depending on the condition and volume of the patient's mastoid cavity, and based on 1 cm³ of the mastoid cavity, the adipose tissue may be contained in an amount of 40 to 80 μl, the three-dimensional PCL scaffold may have a diameter of 3 to 8 mm and a height of 0.5 to 1.5 mm, and may be contained in an amount of 200 to 400 mg, and the whole plasma may be preferably contained in an amount of 40 to 80 μl.

### EMBODIMENTS

The present invention is described in more detail by providing the following embodiments. These embodiments are intended solely to illustrate the present invention, and it would be apparent to one of ordinary skill in the art that the scope of the present invention is not to be construed as limited by these embodiments.

### Embodiment 1: Stemness Analysis of Subcutaneous Adipose-Derived Cells

Subcutaneous adipose was harvested from three 7-week-old male SD mice. The harvested adipose tissues were washed with 1% penicillin/streptomycin-containing PBS, then treated with 100 U/mL collagenase type I in an incubator at 37° C. for 1-2 hours to isolate single cells from the tissues and centrifuged to obtain stromal vascular fraction (SVF) cells. These were then cultured in 1 g/L glucose DMEM medium. To determine the adult stem cell properties of adipose-derived cells on day 6 of culture, fluorescence-activated cell sorting (FACS) analysis was performed using the mesenchymal stem cell markers CD29 and CD90 (BioLegend, San Diego, CA, USA).

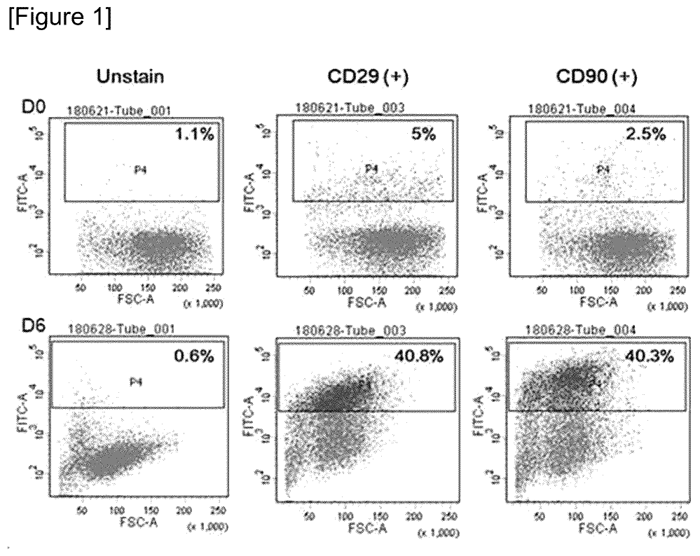

As a result, as shown in FIG. 1, the stemness of CD29- and CD90-expressing cells was identified in the cultured cells on day 6 to 40.8% and 40.3%, respectively, confirming that the adipose tissue-derived cells had the properties of adult stem cells.

### Embodiment 2: Analysis of the Osteogenic Differentiation-Promoting Effects of Plasma Components

To obtain osteogenic differentiation-promoting factors from blood, plasma was collected from the abdominal aorta and centrifuged (400 xg, 15 min) from whole blood to obtain plasma having a 10% volume of whole blood. Platelet-rich plasma and platelet-poor plasma, which are widely used in tissue regeneration, were also collected at 10% volume to determine the osteogenic differentiation-promoting effect on stem cells as a comparison group.

Alizarin Red S and Real-Time qPCR were used to analyze the osteogenic differentiation effect of different post-treatment methods of blood added as an osteogenic differentiation promoter of subcutaneous adipose-derived stem cells.

Subcutaneous adipose-derived stem cells were cultured in a bone differentiation medium environment (low glucose Dulbecco's Modified Eagle's Medium [10% vol./vol. fetal bovine serum, 1% vol./vol. Penicillin/Streptomycin] supplemented with L-Ascorbic acid 2-phosphate [50.0 μM final], Dexamethasone [0.1 μM final] and B-glycerophosphate [10.0 mM final]) depending on the post-treatment conditions of plasma-derived components (10% [vol./vol] platelet-poor plasma [PPP], 10% [vol./vol/] platelet-rich plasma [PRP], 10% [vol./vol.] plasma [whole plasma, WP]) for days 1-14, and the expression level of the bone differentiation marker BMP2 (primer forward: GAAGCCAGGTGTCTCCAAGAG (SEQ ID NO: 1); reserve: GTGGATGTCCTTTACCG TCGT (SEQ ID NO: 2), RUNX2 (forward: GCCGGGAATGATGAGAACTA (SEQ ID NO: 3); reverse: GGACCGTCCACTG TCACTTT (SEQ ID NO: 4), COL1 (forward: CTGCCCAGAAGAATATGTATCACC (SEQ ID NO: 5); reverse: GAAGCAAA GTTTCCTCCAAGACC (SEQ ID NO: 6)), OCN (forward: TGAGGACCCTCTCTGCTC (SEQ ID NO: 7); reserve: AGGT AGCGCCGGAGTCTACC (SEQ ID NO: 8)) was confirmed by Real-Time qPCR, and the degree of mineralization of adipose-derived stem cells was compared by Alizarin red S staining.

The results showed that mineralization of mesodermal stem cells was most effective when provided with plasma-containing osteogenic medium. On day 7 of culture, mineralization was higher when the osteogenic medium (OM) was treated together with plasma, and by day 21 of culture, mineralization occurred in control medium (CM) rather than osteogenic medium.

### Embodiment 3: Analysis of the Effects of a Three-Dimensional Porous Scaffold and Adipose Tissues on Mastoid Bone Regeneration

After the animals (Sprague-Dawley rats; SD-rats) were anesthetized, an anterior skin incision was made, the subcutaneous tissue was debrided to expose the outer wall of the mastoid, and a 3×3 mm perforation was made in the outer wall of the mastoid using a drill.

The mastoid bone defect model was divided into (1) control group (PCL only), (2) PCL scaffold+subcutaneous adipose group, (3) PCL scaffold+subcutaneous adipose group+platelet poor plasma (PPP), (4) PCL scaffold+subcutaneous adipose group+platelet rich plasma (PRP), (5) PCL scaffold+subcutaneous adipose group+plasma (WP; Whole plasma), and (6) PCL scaffold+Graphene oxide (GO)+subcutaneous adipose group+plasma. Transplantation was performed according to the conditions of each group, and the amount of subcutaneous adipose was 200 mg.

Micro-CT, 3D imaging software, Hematoxylin & Eosin staining, and immunochemical staining (Osteocalcin; OCN, Bone sialo-protein; BSP) were used to monitor the development and extent thereof of new bone tissue in the mastoid cavity.

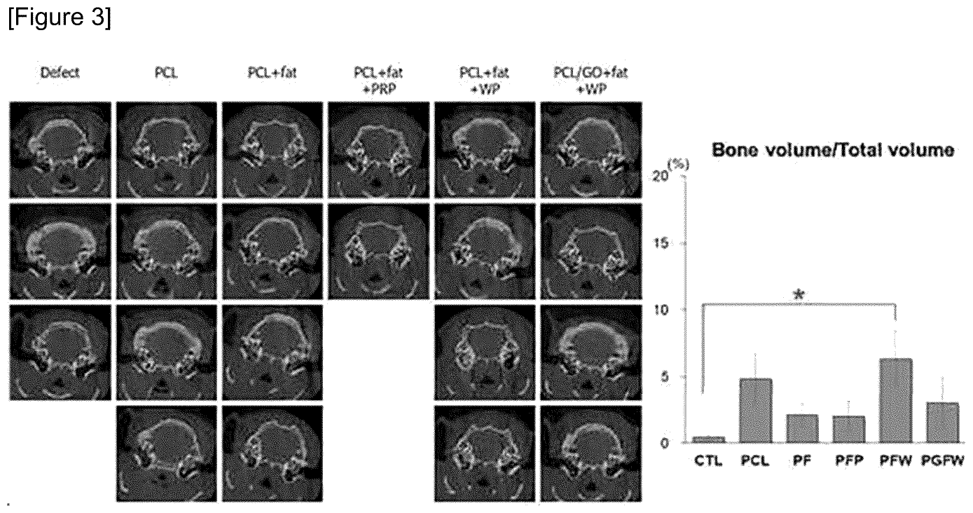

Analysis of CT images of CT images of 2, 4, and 7 months after adipose transplantation to mastoid bone defect models, and H&E, and immunostaining showed that mastoid bone regeneration was observed in (1) the control group (PCL only) and (5) the PCL scaffold+subcutaneous adipose group+whole plasma (WP) group (FIG. 3). To quantitatively evaluate this effect in the effect group and the control group, micro CT and 3D image evaluation were further performed.

As a result, as shown in FIG. 3, the bone regeneration effect was best in the group treating PCL with whole plasma and the group treated with only PCL when comparing the bone volume of newly created bone compared to the total volume.

### Embodiment 4: Validation of Mastoid Bone Regeneration by Immunochemical Staining

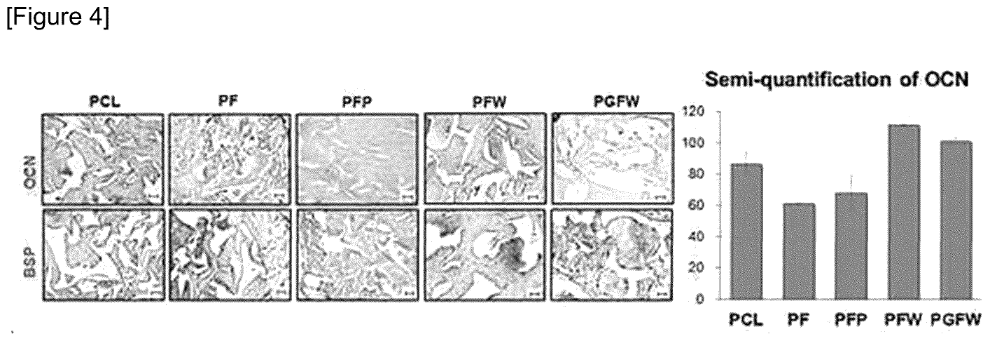

Osteocalcin (OCN) and Bone sialo-protein (BSP), which are used as markers of bone differentiation, were subjected to DAB (3,3′-diaminobenzidine) staining, and the results of tissue staining by group were quantified through Image J. As shown in FIG. 4, it was confirmed that the inclusion of whole plasma was most effective for bone regeneration.

### Industrial Availability

The three-dimensional porous scaffold, adipose-derived cells, and plasma of the present invention have an effect of significantly increasing bone regeneration for mastoid bone defects, and since the adipose tissues and whole plasma obtained as a by-product of mastoid surgery are used for bone regeneration, the mastoid bone regeneration effect can be conveniently achieved in one step during surgery.

While the foregoing has described in detail certain aspects of the present invention, it will be apparent to one of ordinary skill in the art that these specific descriptions are merely preferred embodiments and are not intended to limit the scope of the present invention. Accordingly, the substantial scope of the present invention is defined by the appended claims and their equivalents.

1. A method of regenerating mastoid bone in a subject in need thereof comprising treating a composition containing a three-dimensional porous scaffold and adipose tissues to a mastoid cavity of the subject.

2. The composition method according to claim 1, wherein the scaffold is polycaprolactone (PCL) or graphene.

3. The method according to claim 1, wherein the adipose tissue is autologous adipose tissue.

4. The method according to claim 1, which wherein the composition further comprises plasma.

5. The method according to claim 4, wherein the plasma is autologous plasma.

6. The method according to claim 4, wherein the plasma is whole plasma.
